(19) United States
(12) Patent Application Publication
(10) Pub. No.: US 2025/0041562 A1
(43) Pub. Date: Feb. 6, 2025

(54) INFUSION SYSTEM

(71) Applicant: B. Braun Melsungen AG, Melsungen (DE)

(72) Inventors: Holger Sauer, Waltrop (DE); Tobias Schroeder, Melsungen (DE); Jens Wildhagen, Hannover (DE)

(21) Appl. No.: 18/791,856

(22) Filed: Aug. 1, 2024

(30) Foreign Application Priority Data
Aug. 3, 2023 (DE) 10 2023 120 613.3

(51) Int. Cl. A61M 25/00

(52) U.S. Cl. CPC A61M 25/0043; A61M 2202/0007; A61M 2202/04; A61M 2205/0216; A61M 2205/10; A61M 2205/3331; A61M 2205/50

(57) ABSTRACT

An infusion system includes a catheter with a proximal end, distal end, at least one lumen between the proximal end and distal end, and outlets. Each outlet connects to the at least one lumen to convey fluid, and extends radially through a catheter wall of the catheter. The outlets are spaced apart from in the proximodistal direction, and include a first outlet and a second outlet. The first outlet is opened independently of a fluid pressure prevailing in the lumen in order to administer an analgesic. The second outlet is adapted for fluid pressure-dependent administration of the analgesic and capable of being converted, as a function of fluid pressure inside the lumen, between an opening state, in which the second outlet is opened to administer the analgesic, and a closure state, in which the second outlet is closed. The infusion system can be used in pain management.

### CROSS-REFERENCE TO RELATED APPLICATION

This application claims priority under 35 U.S.C. § 119 to German Application No. 10 2023 120 613.3, filed on Aug. 3, 2023, the content of which is incorporated by reference herein in its entirety.

### FIELD

The present disclosure relates to an infusion system for use in pain management, comprising a catheter with a proximal end, a distal end, at least one lumen, which is elongated between the proximal end and the distal end, and with a plurality of outlets, the plurality of outlets each being connected to the at least one lumen in such a way as to convey fluid, and the plurality of outlets each extending radially through a catheter wall of the catheter and being spaced apart from one another in the proximodistal direction.

### BACKGROUND

Such an infusion system is widely known in medical technology, and is used in the scope of pain management to administer an analgesic. In order to be able to administer the analgesic in such a way that it is distributed between a plurality of locations, the catheter has a plurality of outlets arranged spaced apart from one another in the proximodistal direction. The positioning of the outlets defines the local distribution of the analgesic.

### SUMMARY

It is an object of the present disclosure to provide an infusion system of the type mentioned in the introduction, which enables improved pain management.

This object is achieved in that the plurality of outlets comprise at least one first outlet and at least one second outlet, wherein the first outlet is opened independently of a fluid pressure prevailing in the lumen in order to administer an analgesic, and wherein the second outlet is adapted for fluid pressure-dependent administration of the analgesic and is capable of being converted as a function of the fluid pressure prevailing inside the lumen between an opening state, in which the second outlet is opened in order to administer the analgesic, and a closure state, in which the second outlet is closed. The solution according to the present disclosure enables pressure-dependent control of the administration of the analgesic through the at least second outlet. Since the plurality of outlets and therefore the at least one first outlet and the at least one second outlet are spaced apart from one another in the proximodistal direction, i.e. along the longitudinal axis of the catheter, the position of the analgesic administration can thereby be influenced. The present disclosure is based on the idea that analgesic administration that can be controlled over the length of the catheter may be advantageous in medical terms. The solution according to the present disclosure enables such control in that the analgesic administration through the at least one second outlet can be controlled as a function of the fluid pressure prevailing in the at least one lumen. For this purpose, the at least one second outlet is adapted for fluid pressure-dependent administration of the analgesic. In one embodiment, the at least one second outlet is specially configured for this purpose, for example in the form of a slit or the like. In a further embodiment, the at least one second outlet is assigned a valve for pressure-dependent control. In contrast to the at least one second outlet, the at least one first outlet is not controllable, and in particular is always opened. The at least one second outlet is closed, in particular fully, in the closure state. In one embodiment, the at least one second outlet is not fully closed in the closure state, so that analgesic administration takes place with a reduced flow rate compared with the opening state. Preferably, the at least one second outlet is configured in such a way that a flow rate administered through the at least one second outlet in the opening state corresponds to a flow rate through the at least one first outlet.

In one embodiment, the second outlet is formed by a slit in the catheter wall, the catheter wall being resiliently flexible at least in the region of the slit, the slit being closed at a first fluid pressure and the slit being opened at a higher second fluid pressure, as a result of a resilient deformation of the catheter wall caused by the second fluid pressure. By forming the second outlet as a slit, the fluid pressure-based controllability is achieved with particularly simple means. The slit reaches radially from an inner side of the catheter wall, facing toward the lumen, to an outer side of the catheter wall, facing toward the environment. By the resiliently flexible configuration of the catheter wall at least in the region of the slit, the slit can open and close as a result of a fluid pressure-based resilient deformation of the catheter wall. When the first fluid pressure is applied, the slit is closed and the catheter wall is substantially undeformed in the region of the slit. When the second fluid pressure, which is higher in comparison with the first fluid pressure, is applied, a resilient deformation of the catheter wall takes place in the region of the slit so that the latter opens and enables administration of analgesic through the slit. In one embodiment, the slit is entirely elongated in a straight line, for example in the proximodistal longitudinal direction of the catheter or transversely with respect to the longitudinal direction, particularly in the circumferential direction. In a further embodiment, the slit is elongated in a curve. In a further embodiment, the slit comprises a plurality of differently oriented slit portions that are connected to one another. By selecting the length and/or shaping of the slit as well as the resilient properties of the catheter wall in the region of the slit, the opening and closing behavior may be adapted with simple means to different requirements. By the resilient configuration of the catheter wall at least in the region of the slit, the latter returns automatically from the opening state to the closure state when the fluid pressure is reduced.

In a further embodiment, the slit is elongated in the proximodistal direction and is widened in circumferential direction of the catheter wall in the opening state. Preferably, the slit is entirely elongated in a straight line. By the proximodistally elongated alignment of the slit, an improved opening and closing behavior as a function of the prevailing fluid pressure may be achieved. This is the case particularly in comparison with an alignment of the slit in the circumferential direction of the catheter wall, which may be envisioned in principle.

In a further embodiment, the slit is introduced into the catheter wall in a U-shape in a radial viewing direction and delimits a wall portion, which is pressed radially outward relative to the rest of the catheter wall in the opening state. The wall portion functions in the manner of a resilient tongue or flap. When the first fluid pressure is applied, the wall portion is flush with the rest of the catheter wall, so that the slit is closed. When the higher second fluid pressure is applied, the wall portion is pressed radially outward as a result of the action of pressure, so that the slit is opened. By the resiliently flexible configuration of the catheter wall at least in the region of the slit, the wall portion returns from the opening state automatically to the closure state when the pressure is reduced.

In a further embodiment, the second outlet is assigned a valve, the valve being adapted for fluid pressure-dependent opening and closing, and the valve being closed at a first fluid pressure and opened at a higher second fluid pressure. The valve may have any design suitable for the present purpose. The valve opens and closes automatically as a function of the fluid pressure, for example in the manner of a check valve.

In a further embodiment, the valve is upstream of the second outlet. For example, the valve may be arranged in the lumen or a branch of the lumen, which is assigned to the second outlet. In a further embodiment, the valve is downstream of the second outlet.

In a further embodiment, the at least one first outlet is arranged in the proximodistal direction, in particular centrally, between at least two second outlets. The two second outlets are therefore arranged on either side of the at least one first outlet in relation to the longitudinal axis of the catheter. This may also be described as a peripheral arrangement of the at least two second outlets. This embodiment enables control of the analgesic administration in a peripheral region. Conversely, the administration in the region of the first outlet between the at least two second outlets takes place independently of the prevailing fluid pressure, in particular constantly. In an alternative embodiment, the at least one second outlet is arranged in the proximodistal direction, in particular centrally, between the at least two first outlets. In this embodiment, analgesic administration that does not vary peripherally as a function of the fluid pressure takes place, while fluid pressure-controlled administration can take place through the at least one second outlet in a central region between the two first outlets.

In a further embodiment, a group of a plurality of, in particular precisely three, first outlets is arranged in the proximodistal direction, in particular centrally, between two groups respectively of a plurality of, in particular precisely three, second outlets. By a grouped arrangement respectively of a plurality of first or second outlets, the local distribution of the analgesic administration may be controlled in an improved way. In an alternative embodiment, a group of a plurality of, in particular precisely three, second outlets is arranged in the proximodistal direction, in particular centrally, between two groups respectively of a plurality of, in particular precisely three, second outlets.

In a further embodiment, a delivery device and a control device are provided, the delivery device being connected to the at least one lumen of the catheter in such a way as to convey fluid, and being adapted to deliver the analgesic through the at least one lumen with different fluid pressures, and the control device being connected to the delivery device and being adapted to control the fluid pressure and therefore to open and/or close the at least one second outlet. The delivery device may have any design suitable for the present purpose. For example, the delivery device may be a piston pump, peristaltic pump or elastomer pump, although other designs are of course also conceivable and possible. The control device is adapted to control the delivery device, control of the fluid pressure being provided in particular. By controlling the fluid pressure, the at least one second outlet of the catheter may be opened and/or closed by means of the control device. The opening and/or closing, which is controlled in this way, of the at least one second outlet enables particularly simple local adaptation of the analgesic administration.

### BRIEF DESCRIPTION OF THE DRAWINGS

Further advantages and features of the present disclosure may be found in the following description of preferred exemplary embodiments of the present disclosure, which are represented with the aid of the drawings.

FIG. 1 shows a schematic view of an embodiment of an infusion system according to the present disclosure with a catheter, a delivery device and a control device.

FIGS. 2 and 3 show a portion of the catheter in a radial viewing direction in the region of a second outlet, the second outlet occupying a closed state (FIG. 2) and an opened state (FIG. 3) as a function of a fluid pressure.

FIGS. 4 and 5 show the region of the catheter as shown in FIGS. 2 and 3 with an alternative configuration of the second outlet in the closed state (FIG. 4) and in the opened state (FIG. 5), FIG. 5 showing a longitudinal section with a radial viewing direction rotated through 90°.

FIG. 6 shows a functional diagram to illustrate a further embodiment.

### DETAILED DESCRIPTION

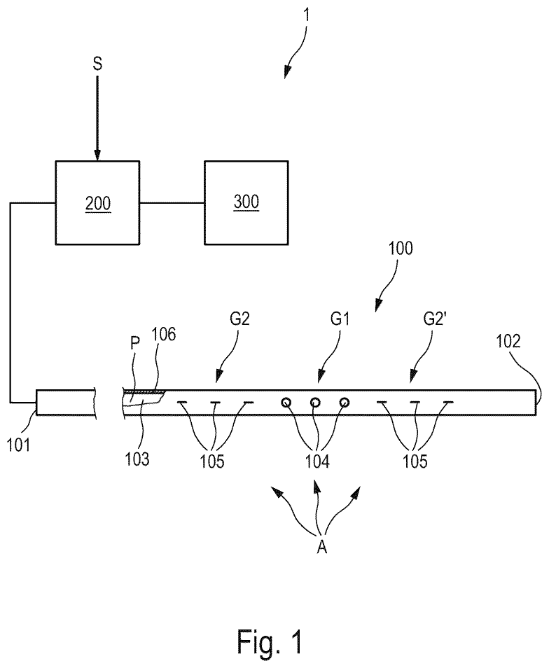

According to FIG. 1, an infusion system 1 for use in pain management is provided, and comprises a catheter 100, a delivery device 200 and a control device 300. The delivery device 200 and/or the control device 300 are optional and are not provided in all embodiments.

The catheter 100, which may also be referred to as an analgesic catheter or pain catheter, comprises a proximal end 101, a distal end 102, at least one lumen 103 and a plurality of outlets A.

The proximal end 101 and the distal end 102 are spaced apart from one another in the proximodistal direction. The catheter 100 is axially elongated between the proximal end 101 and the distal end 102. The entirely straight longitudinal extent shown in FIG. 1 and the shaping shown are to be understood as schematically simplified and purely exemplary.

The lumen 103 is elongated between the proximal end 101 and the distal end 102. In some embodiments, which are not shown in the figures, the catheter comprises a plurality of lumens.

The plurality of outlets A are used to administer an analgesic S. The analgesic S can be delivered by means of the delivery device 200, which is attached (in a manner not shown in detail) to the proximal end 101 in such a way as to convey fluid, into the lumen 103 and from the latter through the outlets A. The outlets A are each connected to the lumen 103 in such a way as to convey fluid. In one embodiment, which is not shown in the figures, different outlets are connected to different lumens of the catheter in such a way as to convey fluid. Further, the outlets A in the present case each extend radially through a catheter wall 106 of the catheter 100 and are spaced apart from one another in the proximodistal direction. In other words, the outlets A are arranged distributed in the longitudinal direction of the catheter 100. This distributed arrangement of the outlets A enables locally distributed administration of the analgesic S.

The arrangement of the plurality of outlets A distributed over the length of the catheter 100, as shown in FIG. 1, and their number, are to be understood as purely exemplary. In some embodiments, which are not shown in the figures, a different number and/or arrangement of the plurality of outlets is provided. In the simplest case, the catheter 100 comprises precisely two outlets.

The plurality of outlets A comprise at least one first outlet 104 and at least one second outlet 105.

In the embodiment shown, a plurality of first outlets 104 and a plurality of second outlets 105 are provided. For simplified description, only a/the first outlet 104 and a/the second outlet 105 will be referred to below. The statements relating to the/a first outlet 104 also apply correspondingly for the further first outlets. The statements relating to the/a second outlet 105 also apply correspondingly for the further second outlets.

The first outlet 104 in the embodiment shown is always opened, so that permanent administration of the analgesic S can take place through the first outlet 104.

The second outlet 105 is adapted for controlled administration of the analgesic S. For this purpose, the second outlet 105 can be converted as a function of a fluid pressure P prevailing inside the lumen 103 between an opening state and a closure state. In the opening state, the second outlet 105 is opened in order to administer the analgesic S. In the closure state, the second outlet 105 is closed. In the embodiment shown, the second outlet is fully closed in the closure state so that no administration, or at least no practically relevant administration, of the analgesic S can take place. In one embodiment, which is not shown in the figures, the second outlet is partially and/or incompletely closed in the closure state, so that administration of the analgesic S can take place with a reduced flow rate compared with the opening state.

By the opening and closing of the second outlet 105 as a function of the fluid pressure P, the administration of the analgesic S can be locally controlled. In the closed state of the second outlet 105, the administration takes place only through the first outlet 104. In the opened state of the second outlet 105, the administration of the analgesic takes place through the first outlet 104 and the second outlet 105.

The fluid pressure P is generated by means of the delivery device 200 and is controlled by means of the control device 300. The local analgesic administration is therefore controlled indirectly by controlling the fluid pressure P.

The delivery device 200 and the control device 300 are schematically represented in FIG. 1. The delivery device 200 is attached to a source (not shown in detail) of the analgesic S. The delivery device 200 may have any design suitable for the present purpose. For example, the delivery device 200 may be configured as a piston pump, peristaltic pump or elastomer pump.

In the embodiment shown, a group G1 of a plurality of first outlets 104 is provided. Furthermore, two groups G2, G2′ respectively of a plurality of second outlets 105 are provided. In the embodiment shown, the groups G1, G2, G2′ each comprise precisely three first outlets 104 and precisely three second outlets 105, respectively. In one embodiment, which is not shown in the figures, different numbers thereof are provided. The group G1 of the first outlets 104 is arranged in proximodistal direction between the two groups G2, G2′ of the second outlets 105. This arrangement is central in the present case, so that the groups may also be described as a central group G1 and two peripheral groups G2, G2′. The embodiment shown in FIG. 1 enables control of the peripheral analgesic administration, while the analgesic administration in the central region of the group G1 is not controllable as a function of the fluid pressure.

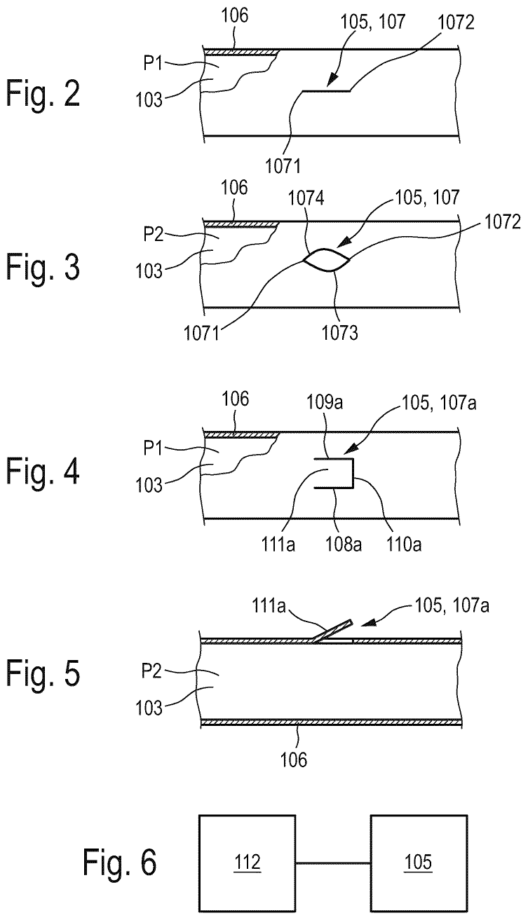

FIGS. 2 to 6 show variants of different configurations of the second outlet 105. The configurations shown respectively allow fluid pressure-dependent control of the second outlet 105 between the opening state and the closure state. The configurations shown in FIGS. 2 to 6 may be provided as alternatives or in combination. For example, the second outlets 105 of group G2 may have a different configuration than the plurality of second outlets 105 of group G2′.

In the variant shown in FIGS. 2 and 3, the second outlet 105 is formed by a slit 107. The catheter wall 105 is resiliently flexible at least in the region of the slit 107. When a first fluid pressure Pl is applied inside the lumen 103, the slit 107 is closed (FIG. 2), i.e. the second outlet 105 occupies the closure state. When a higher second fluid pressure P2 in comparison with the first fluid pressure Pl is applied inside the lumen 103, the slit 107 is opened as a result of a resilient deformation of the catheter wall 106 caused by the second fluid pressure P2 (see FIG. 3). When the fluid pressure decreases from the second fluid pressure P2, the slit 107 closes automatically as a result of the resilient properties of the catheter wall 106.

In the present case, the catheter wall 106 is made from a resilient plastic. Any plastic suitable for the present purpose, with resilient properties and compatibility with medical applications, may be envisioned. Such plastics are known to a person skilled in the art.

In the variant shown in FIGS. 2 and 3, the slit 107 is elongated in the proximodistal direction. The elongation is straight and parallel to the longitudinal axis of the catheter 100. The slit 107 is entirely elongated in a straight line between a proximal slit end 1071 and a distal slit end 1072. In the radial direction, the slit extends from an inner side (not denoted) of the catheter wall 106, facing toward the lumen 103, to an outer side (not denoted) of the catheter wall 106, facing toward the environment. In the opening state (FIG. 3), the slit 107 is widened in the circumferential direction of the catheter wall 106. In this widened state, inner faces 1073, 1074 of the slit 107 that lie opposite one another in the circumferential direction are lifted apart so that the outlet 105 is uncovered. In the closure state (FIG. 2), the inner faces 1073, 1074 bear on one another in a fluid-tight manner, i.e. the second outlet 105 is closed.

In the variant shown in FIGS. 4 and 5, the slit 107a is introduced into the catheter wall 106 in a U-shape in the radial viewing direction of FIG. 4. The U-shaped slit 107a delimits a wall portion 111a of the catheter wall 106. The wall portion 111a is pressed or folded radially outward relative to the rest of the catheter wall 106 in the opening state (FIG. 5). In this way, the second outlet 105 is uncovered. In the closure state (FIG. 4), the wall portion 111a lies substantially flush with the rest of the catheter wall 106 so that the second outlet 105 is closed. The wall portion 11a forms a kind of flap or tongue, which is resiliently mobile relative to the surrounding catheter wall 106 as a function of the prevailing fluid pressure. When the second fluid pressure P2 is applied, the wall portion 11a springs resiliently outward to uncover the second outlet 105. When the fluid pressure decreases from the second fluid pressure P2, the wall portion 11a springs automatically back into the closed state because of the resilient properties of the catheter wall 106.

In the variant shown in FIGS. 4 and 5, the slit 107a comprises a first slit portion 108a, a second slit portion 109a and a third slit portion 110a. The first slit portion 108a and the second slit portion 109a are each elongated in the proximodistal direction and spaced apart from one another in the circumferential direction. In the present case, the first slit portion 108a and the second slit portion 109a are parallel. Furthermore, the first slit portion 108a and the second slit portion 109a are of equal length. The third slit portion 110a is elongated in the circumferential direction and arranged at a distal end respectively of the two slit portions 108a, 109a. The third slit portion 110a connects a distal end (not denoted) of the first slit portion 108a to a distal end (not denoted) of the second slit portion 109a. At an opposite end of the first slit portion 108a and of the second slit portion 109a from the third slit portion 110a, the wall portion 111a delimited by the U-shaped slit 107a is connected to the rest of the catheter wall 106a. This connection forms a resilient flexure bearing or resilient hinge, the imaginary articulation axis in the present case being oriented parallel to the circumferential direction of the catheter wall 106.

In the variant shown in FIG. 6, the second outlet 105 is assigned a valve 112. The second outlet 105 and the valve 112 are schematically represented in a simplified manner in FIG. 6. The valve 112 is adapted for fluid pressure-dependent opening and closing, and is closed when the first fluid pressure P1 is applied and opened when the second fluid pressure P2 is applied. The valve 112 may also be referred to as a two-way valve. The valve 112 may have any design suitable for the present purpose, and may be connected either upstream or downstream of the second outlet 105. Moreover, it is conceivable for the valve 112 itself to form or comprise the second outlet 105.

1. An infusion system for use in pain management, the infusion system comprising:
a catheter with a proximal end, a distal end, at least one lumen elongated between the proximal end and the distal end, and a plurality of outlets,
the plurality of outlets each being connected to the at least one lumen in such a way as to convey fluid, and the plurality of outlets each extending radially through a catheter wall of the catheter and being spaced apart from one another in a proximodistal direction,
the plurality of outlets comprising at least one first outlet and at least one second outlet,
the at least one first outlet being opened independently of a fluid pressure prevailing in the at least one lumen in order to administer an analgesic, and
the at least one second outlet being adapted for fluid pressure-dependent administration of the analgesic and capable of being converted as a function of the fluid pressure prevailing inside the at least one lumen between an opening state, in which the at least one second outlet is opened in order to administer the analgesic, and a closure state, in which the at least one second outlet is closed.

2. The infusion system according to claim 1, wherein the at least one second outlet is formed by a slit in the catheter wall, the catheter wall being resiliently flexible at least in a region of the slit, the slit being closed at a first fluid pressure and the slit being opened at a second fluid pressure that is higher than the first fluid pressure, as a result of a resilient deformation of the catheter wall caused by the second fluid pressure.

3. The infusion system according to claim 2, wherein the slit is elongated in the proximodistal direction and is widened in circumferential direction of the catheter wall in the opening state.

4. The infusion system according to claim 2, wherein the slit is introduced into the catheter wall in a U-shape in a radial viewing direction and delimits a wall portion of the catheter wall, which is pressed radially outward relative to a remainder of the catheter wall in the opening state.

5. The infusion system according to claim 1, wherein the at least one second outlet is assigned a valve, the valve being adapted for fluid pressure-dependent opening and closing, and the valve being closed at a first fluid pressure and opened at a higher second fluid pressure.

6. The infusion system according to claim 5, wherein the valve is upstream of the at least one second outlet.

7. The infusion system according to claim 1, wherein:
the at least one second outlet comprises at least two second outlets, and wherein the at least one first outlet is arranged in the proximodistal direction between the at least two second outlets, or
the at least one first outlet comprises at least two first outlets, and wherein the at least one second outlet is arranged in the proximodistal direction between the at least two first outlets.

8. The infusion system according to claim 7, wherein:
the at least one first outlet comprises a plurality of first outlets, the at least one second outlet comprises two groups of second outlets, and the plurality of first outlets are arranged in the proximodistal direction between the two groups of second outlets, or
the at least one second outlet comprises a plurality of second outlets, the at least one first outlet comprises two groups of first outlets, and the plurality of second outlets are arranged in the proximodistal direction between the two groups of first outlets.

9. The infusion system according to claim 1, further comprising:
a delivery device; and
a control device,
the delivery device being connected to the at least one lumen of the catheter in such a way as to convey fluid, and being adapted to deliver the analgesic through the at least one lumen with different fluid pressures, and
the control device being connected to the delivery device and being adapted to control the fluid pressure and therefore to open and/or close the at least one second outlet.
